(19) United States
(12) Patent Application Publication
(10) Pub. No.: US 2019/0336037 A1
(43) Pub. Date: Nov. 7, 2019

(54) LIGHT EMITTING SURGICAL CALIBRATION PROBE DEVICE

(71) Applicant: EPED Inc., Kaosiung City 821 (TW)

(72) Inventors: Ta-Ko Huang, Kaohsiung City 821 (TW); Jerry T. Huang, Kaohsiung City 821 (TW)

(21) Appl. No.: 16/402,740

(22) Filed: May 3, 2019

(30) Foreign Application Priority Data
May 4, 2018 (TW) 107115338
May 3, 2019 (TW) 108115411

(51) Int. Cl. A61B 5/06; A61B 90/00

(52) U.S. Cl. CPC A61B 5/064; A61B 5/065; A61B 2090/3945; A61B 2560/0223; A61B 2562/225; A61B 90/39

(57) ABSTRACT

A light emitting surgical calibration probe device includes a first optical tracking assembly, a quick disconnect coupler, a second positioning assistance unit, and a probe. The first optical tracking assembly has at least one tracking markers on a surface, and a surgical navigation system converts the position of the tracking markers into an overall position. The quick disconnect coupler is detachably coupled to the first optical tracking assembly and the second positioning assistance unit, and the second positioning assistance unit is capable of emitting an indicating light source with a specific wavelength and provided for a surgical navigation system to perform a non-contact positioning function. The probe is movably coupled to the second positioning assistance unit. The first optical tracking assembly and the second positioning assistance unit allow doctors to know the position of a surgical robot with respect to a patient's trackable housing more accurately.

### FIELD OF THE INVENTION

The present invention relates to the technical field of a surgical probe device, and more particularly to a light emitting surgical calibration probe device.

### BACKGROUND OF THE INVENTION

In recent years, a surgical navigation system (not shown in the figure) is provided for assisting a doctor in a surgery to locate the position of a surgical robot with respect to a patient's trackable housing, so that the surgical operation can be performed more quickly, accurately, and safely.

With reference to FIG. 1 for a conventional surgical probe device 1 which is applicable to the aforementioned surgical navigation system, the surgical probe device 1 comprises a optical tracking assembly 11, a Tightening screw 12 installed to the optical tracking assembly 11, and a probe assembly 13 coupled to the optical tracking assembly 11. This probe device 1 can be used to complete a positioning operation in a surgery.

Wherein, the optical tracking assembly 11 comprises a trackable housing 111, a plurality of tracking markers 112 disposed on a surface of the trackable housing 111, and an coupler 113 extending from the trackable housing 111. The tracking markers 112 of the optical tracking assembly 11 is provided for the surgical navigation system to perform positioning; the Tightening screw 12 is a screw 12 secured to the coupler 113 of the optical tracking assembly 11; the probe assembly 13 comprises a probe grip 131, and a probe 132 fixed to the probe grip 131, and the probe grip 131 of the probe assembly 13 is abutted against and fixed to the coupler 113 of the optical tracking assembly 11 by the rotation of the tightening screw 12.

The drawback of the prior art resides on the difficulty of installing and removing the probe. In addition, when the tightening screw 12 is packed tightly and fixed to the coupler 113 and the probe grip 131, a slight error may be produced in an image displayed in a surgical navigation due to the error of the rotating depth, thus affecting the proceeding of the surgery. In addition, the calibration of the positioning is performed before the surgery. According to present existing guidelines, any apparatus that has been used before a surgery cannot be used again in an invasive surgery or surgical operation in order to prevent infection. If a doctor needs to perform a positioning operation in a surgical operation, the doctor will need to remove the old probe and then install another probe after disinfection. In other words, the probe is changed after the secured screw is turned and loosened. Obviously, the conventional probe device is very inconvenient.

### SUMMARY OF THE INVENTION

Therefore, it is a primary objective of the present invention to provide a light emitting surgical calibration probe device capable of completing the operation of changing the probe quickly.

To achieve the aforementioned and other objectives, the present invention provides a light emitting surgical calibration probe device comprising: a first optical tracking assembly, a quick disconnect coupler, a second positioning assistance unit and a probe. The first optical tracking assembly has at least one tracking markers disposed on a surface thereof and provided for the surgical navigation system to convert the position of the tracking markers into an overall position. The quick disconnect coupler is detachably coupled to the first optical tracking assembly and the second positioning assistance unit, and the second positioning assistance unit can emit an indicating light source of a specific wavelength to a specified number and is provided for the surgical navigation system to perform a non-contact positioning function, wherein the probe is movably coupled to the second positioning assistance unit.

Further, the first optical tracking assembly comprises a first trackable housing, and a first coupler extending from the first trackable housing, and the quick disconnect coupler is detachably coupled to the first coupler. Therefore, the first optical tracking assembly can be combined stably with the quick disconnect coupler. The second positioning assistance unit comprises a framework, a laser module and a control unit, and the laser module and the control unit are combined with the framework, and the control unit is electrically coupled to the laser module for controlling the laser module, wherein the control unit comprises a switch, and the laser module is capable of emitting an indicating light source with at least one specific wavelength. The framework of the second positioning assistance unit is capable of making the installation of the laser module more stable, and allowing the required structure to be mounted (or integrally formed or welded) onto the framework securely, so as to make more stable and secured

Further, this embodiment also discloses that the at least one tracking markerstracking markers is installed on a surface of the first trackable housing, and the tracking markers is a light emitting element or a light reflecting element. Therefore, the positioning system can capture its position information.

Further, the probe has a thread, and the second positioning assistance unit has a corresponding threaded hole, so that the thread can be screwed and combined into the threaded hole.

Further, the second positioning assistance unit comprises a second trackable housing, and a probe grip extending from the second trackable housing.

Further, the second positioning assistance unit emits an indicating light source with at least one specific wavelength, and the indicating light source is provided for the positioning system to recognize or for the user to view, and the indicating light source may be a visible light or an invisible light as needed. To cope with the system status, the visible and invisible indicating light sources of a specific wavelength can be emitted synchronously, and these indicating light sources can be emitted coaxially.

The present invention has the effects of changing the probe quickly by a quick release and improving the convenience of operation in a surgery. In addition, the installation of the second positioning assistance unit facilitates doctors to carry out the non-contact positioning. In other words, the present invention has both contact and non-contact positioning function, which is the pioneering work in the related field.

### BRIEF DESCRIPTION OF THE DRAWINGS

FIG. 1 is a schematic view of a conventional surgical probe;

FIG. 2 is an exploded view of an embodiment of the present invention;

FIG. 3 is a schematic view of a first optical tracking assembly arranged at different angles in accordance with an embodiment of the present invention; and

FIG. 4 is a schematic view of combining a quick disconnect coupler at different angles in accordance with an embodiment of the present invention.

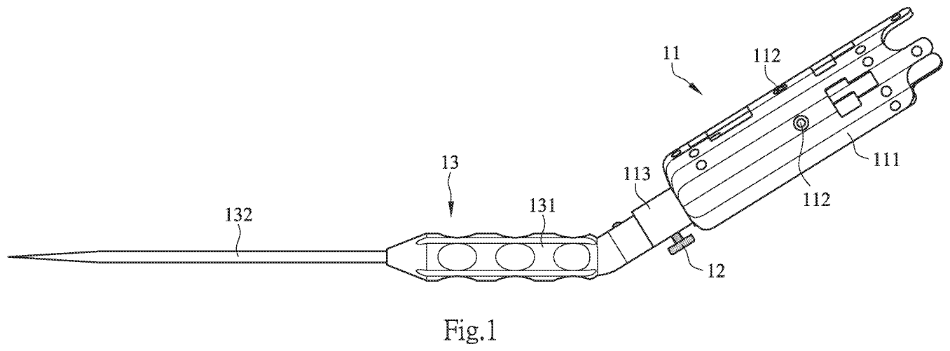

### DESCRIPTION OF THE PREFERRED EMBODIMENTS

The technical contents of the present invention will become apparent with the detailed description of preferred embodiments accompanied with the illustration of related drawings as follows. It is intended that the embodiments and figures disclosed herein are to be considered illustrative rather than restrictive.

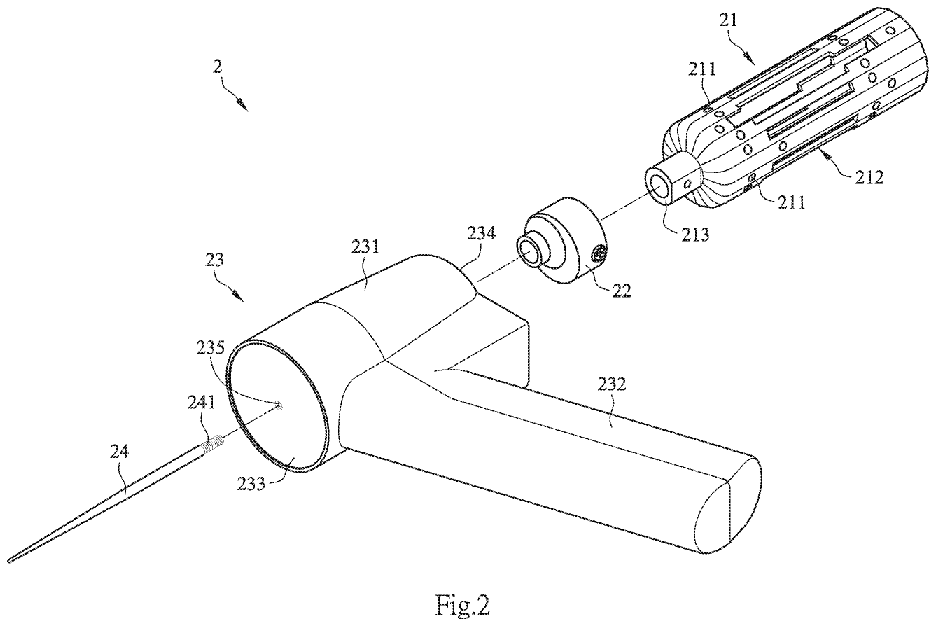

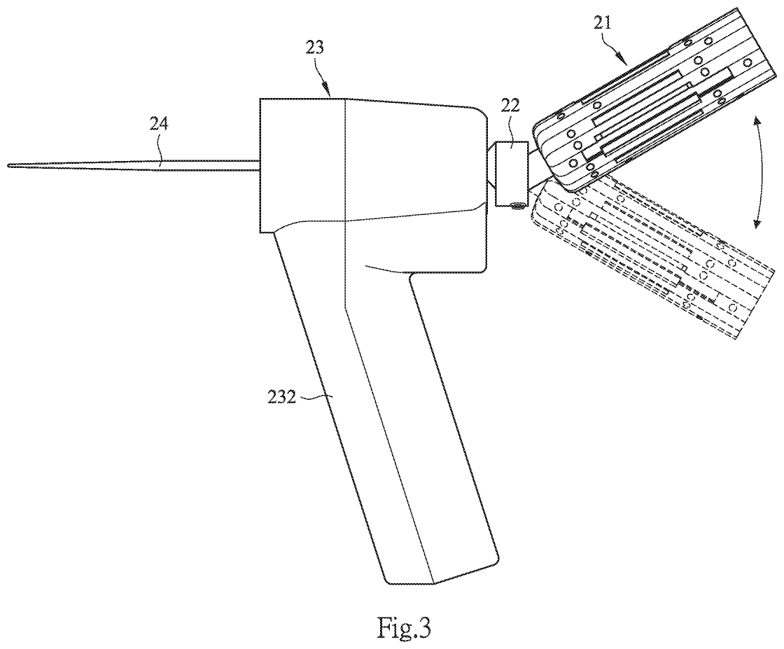

With reference to FIGS. 2 and 3 for a light emitting surgical calibration probe device 2 in accordance with a preferred embodiment of the present invention, the light emitting surgical calibration probe device 2 is applicable to a surgical navigation system (not shown in the figure).

The light emitting surgical calibration probe device 2 comprises: a first optical tracking assembly 21, a quick disconnect coupler 22, a second positioning assistance unit 23, and a threaded probe 24. Wherein, the quick disconnect coupler 22 is detachably coupled to the first optical tracking assembly 21 and the second positioning assistance unit 23, and the first optical tracking assembly 21 and the second positioning assistance unit 23 are disposed on opposite sides of the quick disconnect coupler 22 respectively. The threaded probe 24 is coupled to the second positioning assistance unit 23 and configured in a direction opposite to the second positioning assistance unit 23.

In addition, the first optical tracking assembly 21 has at least one tracking markers 211 disposed on a surface thereon and provided for the surgical navigation system to perform positioning, and the tracking markers 211 nay be embedded into or protruded from a light reflecting element or a light emitting element of the positioning assistance unit 21, so that a surgical navigation system can capture its position and complete a 3D simulated image.

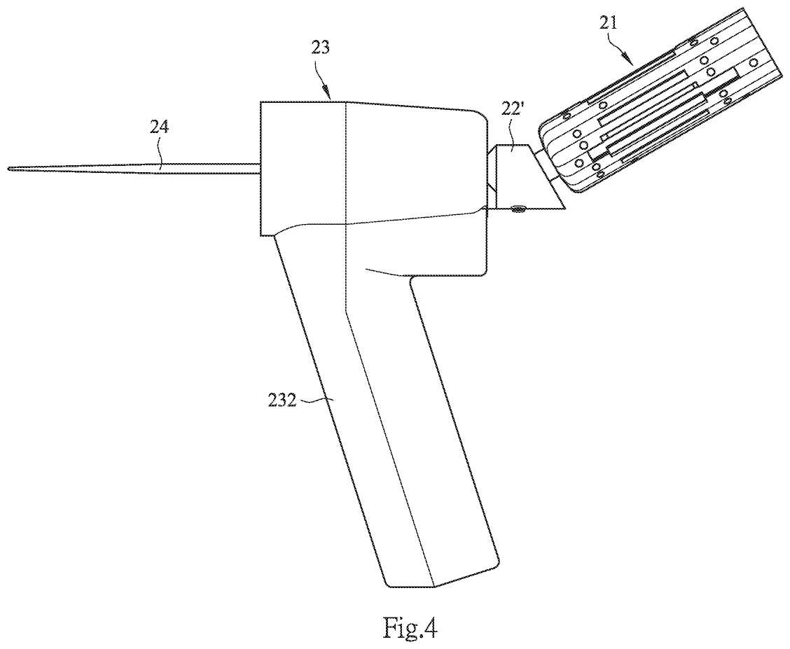

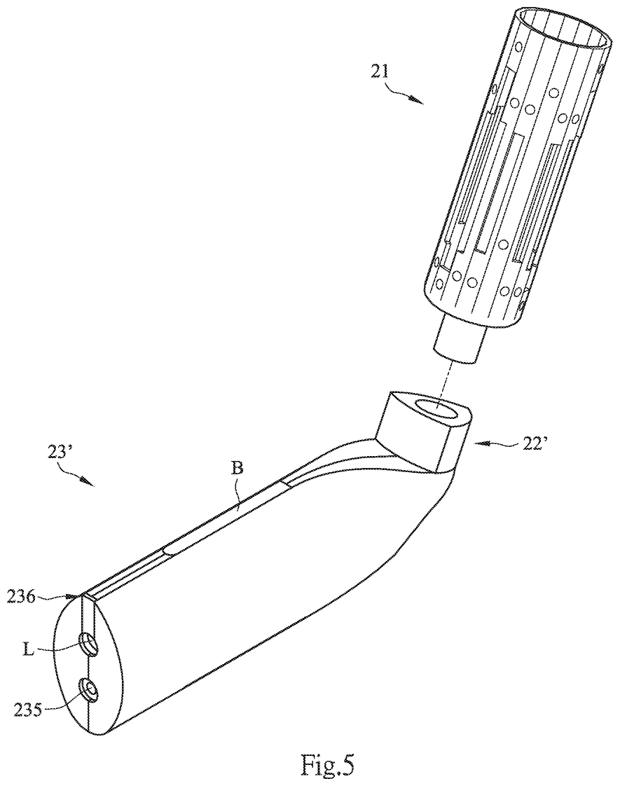

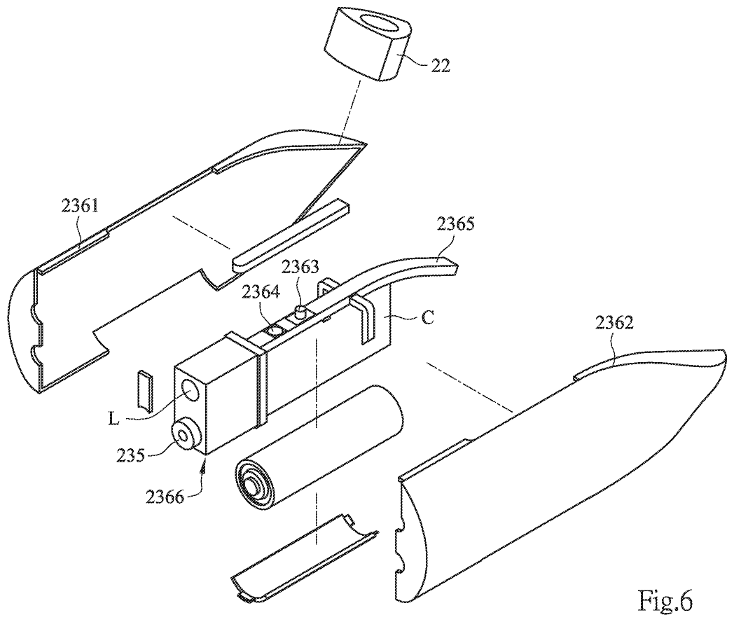

From the description above, this embodiment has the quick disconnect coupler 22 capable of connecting or separating the first optical tracking assembly 21 and the second positioning assistance unit 23 quickly. Wherein, the quick disconnect coupler 22 may further have a mechanism similar to a universal joint, so that the first optical tracking assembly 21 can be rotated with respect to the quick disconnect coupler 22 to change the included angle between the first optical tracking assembly 21 and the second positioning assistance unit 23. During an operation performed by a doctor, the overall movement will not be affected by the position of the first optical tracking assembly 21, and thus the invention can reduce hindrance and maintain the smoothness of a surgical operation. In FIG. 4, the quick disconnect coupler 22′ has another corresponding fixed angle which is provided for the direct selection by the doctor during the operation. In other words, the quick disconnect coupler 22 provides different angles for the doctor, and the doctor may select an appropriate angle for use according to the situation.

The first optical tracking assembly 21 comprises a first trackable housing 212, and a first coupler 213 extending from the first trackable housing 212, and the quick disconnect coupler 22 is detachably coupled to the first coupler 213. In this preferred embodiment, the first trackable housing 212 is in a cylindrical shape, and the at least one tracking markers 211 is installed on a surface of the first trackable housing 212, wherein the tracking markers 211 may be embedded into the first trackable housing 212 or protruded from the first trackable housing 212.

The second positioning assistance unit 23 comprises a second trackable housing 231, and a grip handle 232 extending from the second trackable housing 231. Wherein, the grip handle 232 is provided for a user to hold, and the threaded probe 24 and the quick disconnect coupler 22 are coupled to both ends 233, 234 of the second trackable housing 231 respectively

It is noteworthy that the second positioning assistance unit 23 is capable of emitting an indicating light source with a specific wavelength, and the indicating light source is provided for the surgical navigation system to carry out a non-contact positioning process. The doctor may select the use of contact or non-contact positioning, and thus the invention has the effect of improving the quality and precision of a surgical operation. In addition, there are two or more indicating light sources with different specific wavelengths, but these indicating light sources can emit light coaxially, and one of the indicating light sources is an invisible indicating light source of a specific wavelength (such as the infrared with a wavelength of 850) which is provided for a navigation system to determine a position, and the other indicating light source is a visible infrared (such as a red light spot with a wavelength of 650), which is provided for a user (such as a doctor) to visually recognize the position of the positioning point, so as to improve the overall accuracy of the surgery.

The threaded probe 24 is detachably coupled to the second positioning assistance unit 23, wherein the threaded probe 24 includes a thread 241, and the second positioning assistance unit 23 further includes a corresponding screw hole 235, and the threaded probe 24 can be detachably secured to the second positioning assistance unit 23. Since the threaded probe 24 can be removed independently, therefore the threaded probe 24 can be disinfected directly after the surgery, and the space required for disinfecting the components can be further reduced, and this effect has never been achieved before.

Further, the present invention provides another embodiment of a second positioning assistance unit 23′ as shown in FIGS. 5 and 6

The second positioning assistance unit comprises: a framework 2365, a laser module 2366 installed at a front end of the framework 2365; a laser light exit L formed at a front end of the laser module 2366, wherein the threaded hole 235 is formed at a position adjacent to the laser light exit L for connecting the probe securely; a control unit C coupled to the framework 2365 and electrically coupled to the laser module 2366; a switch 2363; and an electric power indicating lamp 2364. A housing 236 is provided for covering the framework 2365, the control unit C and the laser module 2366, and the housing 236 comprises a left housing 2361 and a right housing 2362, and two openings formed at positions corresponding to the laser light exit L and the threaded hole 235 respectively. In addition, the housing 236 have a movable button B formed thereon and configured to be corresponsive to the switch 2363, so that a user can press the button B to trigger a switch 2363. Of course, the switch 2362 may also be protruded from the housing 236 and provided for the user to operate without the button B.

In addition, the housing 236 may be configured to be corresponsive to a reading window (not shown in the figure) of the electric power indicating lamp 2364, or disposed adjacent to the electric power indicating lamp 2364, and the button B may be made of a translucent material to provide a function for the purpose of reading the power level from the electric power indicating lamp 2364 at the same time.

In addition, the threaded hole 235 is combined and installed to the framework 2365. In other words, the threaded hole 235 extends from the framework 2365 to the laser module 2366 to combine with the laser module 2366, so that the laser module 2366 and the threaded hole 235 can be stabilized at their relative positions. In this embodiment, the framework 2365 is in the shape of a long strip, and a support extends from both sides of the frame work 2362 separately to improve the stability of combining with the housing. The electric power indicating lamp 2364 and the switch 2363 may be installed on the framework 2365.

Further, the quick disconnect coupler 22 for coupling the first optical tracking assembly 21 may be installed at the housing 236 or directly coupled to the framework 2365. If the quick disconnect coupler 22 is coupled to the framework 2365 directly or integrally formed with the framework 2365, the durability and stability of the quick disconnect coupler 22 can be improved.

In summation of the description above, the present invention has the following effects: 1. The first optical tracking assembly 21 and the second positioning assistance unit 23 provide a composite positioning method that allows doctors to operate in many ways to complete positioning and improves the quality and accuracy of the surgery significantly. 2: The design of the quick disconnect coupler 22 provides an easy way to remove the second positioning assistance unit 23 in order to complete the change quickly and decrease the wait time. 3. Since the threaded probe 24 is detachably coupled to the second positioning assistance unit 23, therefore the space required by disinfecting components is reduced. Particularly, the disinfection method of disinfecting components varies, and thus the detachable method can improve the integrity and safety of the disinfection.

What is claimed is:

1. A light emitting surgical calibration probe device, comprising:
a first optical tracking assembly, having at least one tracking markers installed to a surface thereof and provided for a surgical navigation system to perform positioning;
a quick disconnect coupler, detachably coupled to the first optical tracking assembly;
a second positioning assistance unit, coupled to the quick disconnect coupler, comprising a framework, a laser module and a control unit, and the laser module and the control unit being coupled to the framework, and the control unit being electrically coupled to the laser module, for controlling the laser module, wherein the control unit comprises a switch, and the laser module may emit an indicating light source with at least one specific wavelength; and
a probe, movably coupled to the second positioning assistance unit, and disposed on a side opposite to the quick disconnect coupler and on the same side with the laser module.

2. The light emitting surgical calibration probe device according to claim 1, wherein the first optical tracking assembly comprises a first trackable housing and a first coupler extending from the first trackable housing, and the quick disconnect coupler is detachably coupled to the first coupler.

3. The light emitting surgical calibration probe device according to claim 2, wherein the at least one tracking markers is installed on a surface of the first trackable housing, and the tracking markers is a light emitting element or a light reflecting element.

4. The light emitting surgical calibration probe device according to claim 1, wherein the probe has a thread, and the second positioning assistance unit has a corresponding threaded hole, so that the thread may be screwed into and engaged with the threaded hole

5. The light emitting surgical calibration probe device according to claim 1, wherein the second positioning assistance unit comprises a second trackable housing, and a probe grip extending from the second trackable housing.

6. The light emitting surgical calibration probe device according to claim 2, wherein the first trackable housing is substantially in a cylindrical shape.

7. The light emitting surgical calibration probe device according to claim 1, further comprising two or more indicating light sources with a specific wavelength, and at least one of the indicating light sources being a visible indicating light source of a specific wavelength and at least one of the indicating light sources being an invisible indicating light source of a specific wavelength.

8. The light emitting surgical calibration probe device according to claim 7, wherein the indicating light source with each specific wavelength is emitted coaxially
